(19) United States
(12) Patent Application Publication
(10) Pub. No.: US 2015/0216731 A1
(43) Pub. Date: Aug. 6, 2015

(54) EAR WARMER FRAME

(71) Applicant: 180s, Inc., Baltimore, MD (US)

(72) Inventors: Kevin Bouza, Metairie, LA (US); Spencer Stevens, White Marsh, MD (US); Shawn Pinamonti, Parkville, MD (US); Zhiping Yang, Laurel, MD (US)

(21) Appl. No.: 14/174,383

(22) Filed: Feb. 6, 2014

(51) Int. Cl. A61F 11/14

(52) U.S. Cl. CPC A61F 11/14

(57) ABSTRACT

Apparatus described herein include ear warmer frames. In some embodiments, an apparatus includes a frame configured to be disposed about a portion of a head of a user. The frame includes a first member and a second member. The first member has a first portion and a second portion. The first portion of the first member has a cross-sectional profile, and the second portion of the first member has a cross-sectional profile smaller than the cross-sectional profile of the first portion of the first member. An end portion of the second member has a cross-sectional profile substantially corresponding to the cross-sectional profile of the first portion of the first member. The second member defines an opening configured to receive the second portion of the first member such that when the frame is in a collapsed configuration an abutment interface is defined, and surfaces of the abutment interface are substantially continuous between the first member and the second member.

### BACKGROUND

This invention relates generally to an ear warmer frame. More specifically, some embodiments described herein relate to an adjustable ear warmer frame configured to extend around a back of a user's head.

Known ear warmers typically extend over a top of a user's head. Such a known ear warmer typically can have a frame and a layer of fabric on each side of an ear portion of the frame. These layers of fabric and the frame itself typically have a weight appropriate for sedentary outdoor-activities, but not for non-sedentary outdoor-activities such as running or jogging. In other words, when participating in certain outdoor activities, the participant desires apparel that keeps him or her warm while minimizing the weight of such apparel. While capable of keeping the user warm, known ear warmers typically do not have such desired minimal weight. Additionally, the participant may desire to wear apparel during such non-sedentary outdoor activities that fits the participant comfortably and securely. Known ear warmers typically do not provide for both an adjustable fit, which increases comfort of the user, and a secure fit.

Thus, a need exists for an adjustable ear warmer frame that is lightweight while still providing warmth to the user. A need also exists for an adjustable ear warmer frame that can fit a user comfortably and securely.

### SUMMARY

Apparatus described herein include ear warmer frames. In some embodiments, an apparatus includes a frame configured to be disposed about a portion of a head of a user. The frame includes a first member and a second member. The first member has a first portion and a second portion. The first portion of the first member has a cross-sectional profile, and the second portion of the first member has a cross-sectional profile smaller than the cross-sectional profile of the first portion of the first member. An end portion of the second member has a cross-sectional profile substantially corresponding to the cross-sectional profile of the first portion of the first member. The second member defines an opening configured to receive the second portion of the first member such that when the frame is in a collapsed configuration an abutment interface is defined, and surfaces of the abutment interface are substantially continuous between the first member and the second member.

### BRIEF DESCRIPTION OF THE DRAWINGS

FIG. 1 is a perspective view of an ear warmer frame, according to an embodiment.

FIG. 2 is a perspective view of an ear warmer band in a collapsed configuration, according to an embodiment.

FIG. 3 is a perspective view of an ear warmer band in an expanded configuration, according to an embodiment.

FIGS. 4-6 are views of a first band member, according to an embodiment.

FIGS. 7-9 are views of a second band member, according to an embodiment.

FIGS. 10 and 11 are views of the band of FIG. 2 in a collapsed configuration.

FIGS. 12, 13 and 13A are views of the band of FIG. 2 in an expanded configuration.

FIG. 14 is a cross-sectional view of the first band member of FIG. 13 taken along line A-A.

FIG. 15 is a cross-sectional view of the first band member of FIG. 13 taken along line B-B.

FIG. 16 is a cross-sectional view of the ear warmer band of FIG. 13 taken along line C-C.

FIGS. 17 and 18 are perspective views of an ear warmer frame in a collapsed configuration and an expanded configuration, respectively, according to an embodiment.

### DETAILED DESCRIPTION

Apparatus described herein include ear warmer frames. An apparatus according to an embodiment includes a frame configured to be disposed about a portion of a head of a user. The frame includes a first member and a second member. The first member has a first portion and a second portion. The first portion of the first member has a cross-sectional profile, and the second portion of the first member has a cross-sectional profile smaller than the cross-sectional profile of the first portion of the first member. An end portion of the second member has a cross-sectional profile substantially corresponding to the cross-sectional profile of the first portion of the first member. The second member defines an opening configured to receive the second portion of the first member such that when the frame is in a collapsed configuration an abutment interface is defined, and surfaces of the abutment interface are substantially continuous between the first member and the second member.

In some embodiments, the first member includes a first part of a logo and the second member includes a second part of the logo such that a continuous surface of the abutment interface includes the logo when the frame is in the collapsed configuration. In some embodiments, the first member includes a first part of a logo and the second member includes a second part of the logo such that the first part of the logo and the second part of the logo are separated when the frame is in an expanded configuration, and the first part of the logo and the second part of the logo are abutting on a surface of the abutment interface from the surfaces that are substantially continuous when the frame is in the collapsed configuration.

An apparatus according to another embodiment includes a frame configured to be disposed about a portion of a head of a user. The frame includes a first member having a first portion, a second portion, and a shoulder disposed between the first portion and the second portion. The frame also includes a second member having an end surface defining an opening to a channel. The channel is completely defined by the second member. The second portion of the first member is configured to be fully disposed within the channel when the frame is in a collapsed configuration such that the shoulder abuts the second member. A perimeter of the shoulder substantially corresponds to a perimeter of the end surface such that a substantially continuous transition is formed between the first member and the second member when the frame is in the collapsed configuration.

In some embodiments, the second portion of the first member of the frame and a portion of the second member that contains the channel collectively define an intermediate portion of the frame having a substantially uniform radius of curvature when the frame is in an unbiased configuration. In some embodiments, the second portion of the first member is configured to deform when the frame is moved from the unbiased configuration to a biased configuration such that the intermediate portion does not have a uniform radius of curvature when the frame is in the biased configuration.

An apparatus according to another embodiment includes a frame configured to be disposed about a portion of a head of a user. The frame includes a first member having a first portion, a shoulder, and a second portion. The shoulder of the first member is disposed between the first portion of the first member and the second portion of the first member. The second portion has a substantially uniform radius of curvature when the frame is in a collapsed configuration. The frame includes a second member having an end surface defining an opening to a channel. A portion of the second member has (1) a length substantially equal to a length of the second portion of the first member and (2) a substantially uniform radius of curvature substantially equal to the uniform radius of curvature of the second portion of the first member when the frame is in the collapsed configuration. The shoulder abuts the end surface when the frame is in the collapsed configuration. A perimeter of the shoulder substantially corresponds to a perimeter of the end surface such that the frame substantially continuously transitions from the first member to the second member when the frame is in the collapsed configuration.

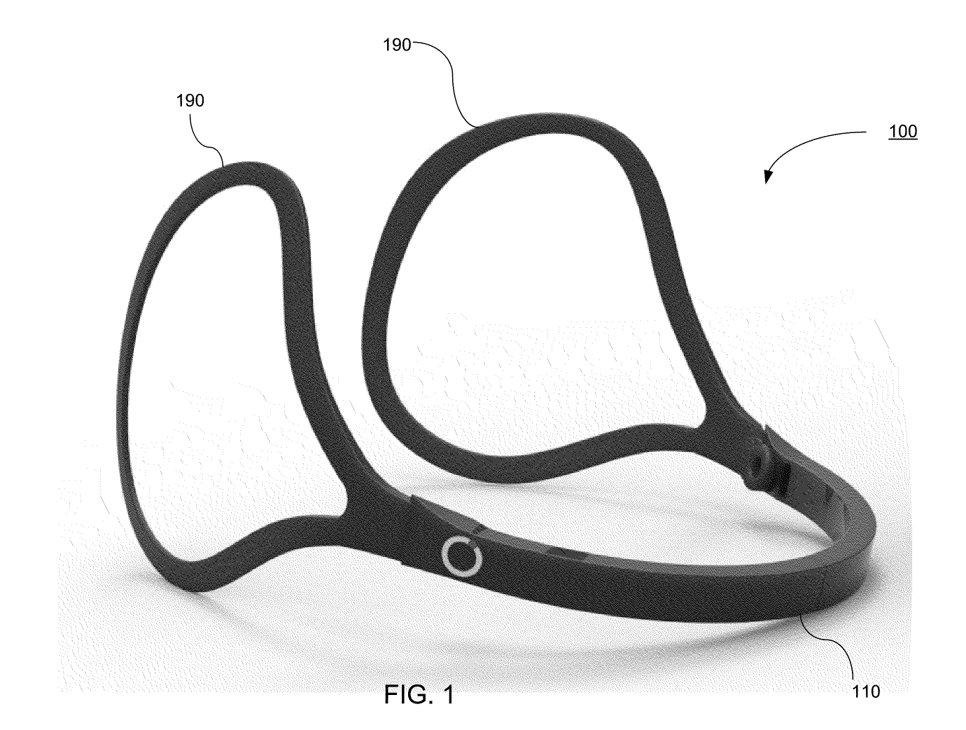

FIG. 1 depicts an ear warmer frame 100 according to an embodiment. The ear warmer frame includes a band 110 and two ear members 190. The ear warmer frame 100 can be similar in many respects to any of the ear warmer frames shown and described in U.S. Pat. No. 7,650,649, issued Jan. 26, 2010, entitled, “Ear Warmer Having an External Frame;” U.S. Pat. No. 7,962,970, issued Jun. 21, 2011, entitled “Ear Warmer Having a Curved Ear Portion;” U.S. patent application No. 12,168,001, filed Aug. 12, 2003, entitled, “Ear Warmer with Fabric Member;” U.S. patent application Ser. No. 12/168,015, filed Aug. 12, 2003, entitled, “Ear Warmer with a Substantially Continuous Surface;” U.S. patent application Ser. No. 12/168,027, filed Aug. 12, 2003, entitled, “Ear Warmer with Fabric Member;” and/or U.S. patent application Ser. No. 13/164,901, filed Jun. 21, 2011 entitled, “Ear Warmer Having a Curved Portion, each of which is incorporated herein by reference in its entirety. For example, a membrane (not shown in FIG. 1) can be removeably coupled to the ear members 190 such that the membrane is disposed on an inner side of the ear warmer frame. As another example, the ear members 190 can be pivotally coupled to the band 110 and moveable between an expanded and a collapsed configuration. Furthermore, the ear members 190 can abut the band 110 when the ear members 190 are in the expanded configuration forming a substantially continuous transition between the ear members 190 and the band 110.

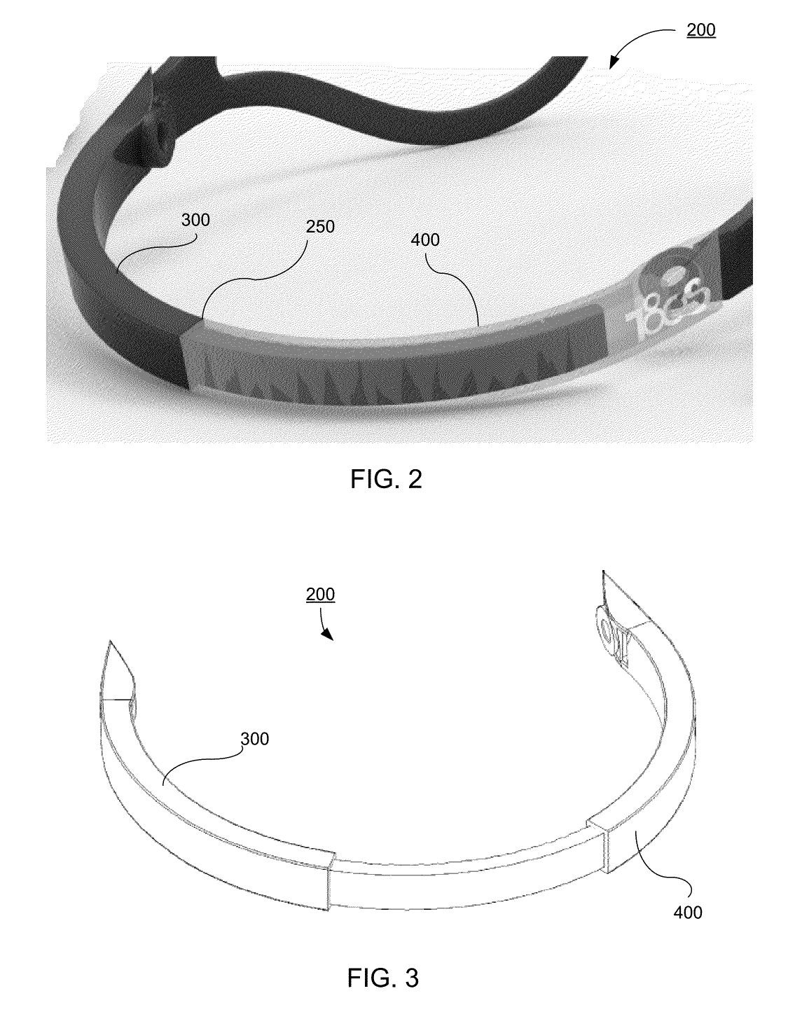

FIG. 2 depicts an ear warmer band 200 of an ear warmer frame according to an embodiment in a collapsed configuration. FIG. 3 depicts the ear warmer band 200 (also referred to herein simply as “band 200”) in an expanded configuration. The band 200 can be structurally and/or functionally similar to the band 110 shown and described above with reference to FIG. 1. The band 200 includes a first band member 300 (also referred to herein as “first member 300”) and a second band member 400 (also referred to herein as “second member 400”). As described in further detail herein, the first band member 300 is slideably coupled to the second band member 400 such that, when the first band member 300 is moved relative to the second band member 400, a length of the band 200 can be adjusted. A portion of the second band member 400 is shown in shadow to show a portion of the first band member 300 disposed within a channel of second band member 400. The band 200 is configured to be movable between a collapsed configuration and an expanded configuration. When the band 200 is in the collapsed configuration, the band 200 has a minimum length. In the collapsed configuration, the first band member 300 abuts the second band member 400 forming a substantially continuous transition at an abutment interface 250. When the band 200 is in the expanded configuration, the band 200 has a length greater than the minimum length, up to a maximum length. The band 200 is adjustable to any suitable length within a range from its minimum length to its maximum length.

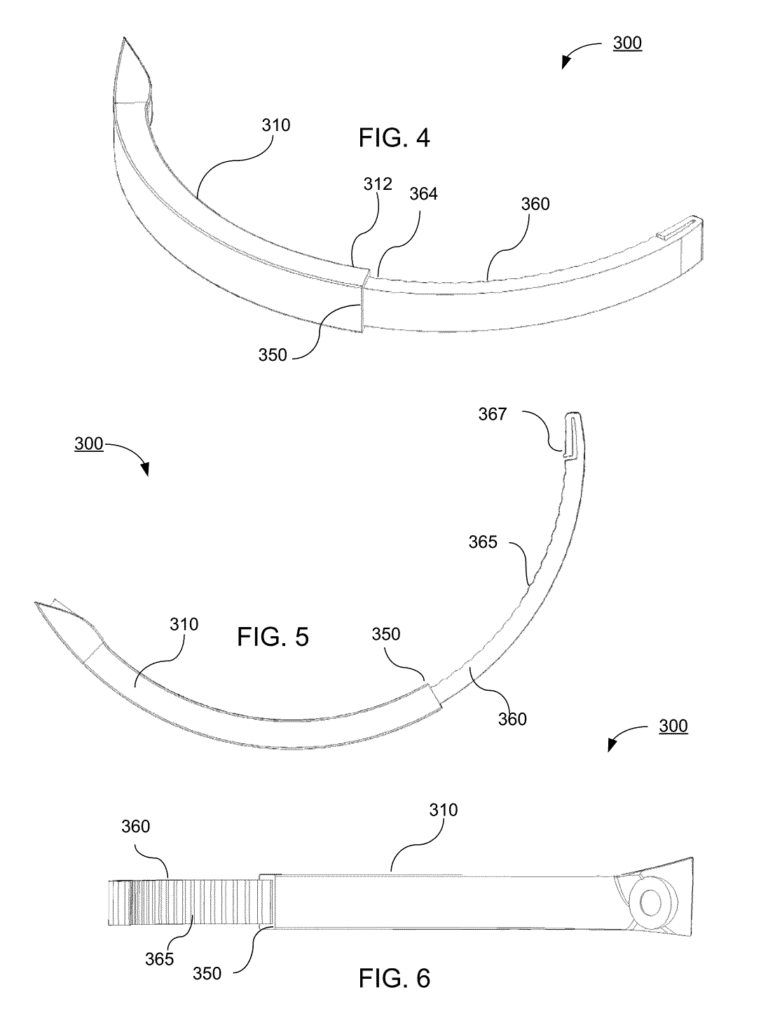

FIGS. 4-6 are views of the first band member 300 (i.e., a perspective view, top view and front view, respectively). The first band member 300 has a first portion 310 and a second portion 360. A first end 312 of the first portion 310 of the first band member 300 is fixed (i.e., non-movable) with respect to a second end 364 of the second portion 360 of the first band member 300. For example, in some embodiments, the first end 312 of the first portion 310 of the first band member 300 is fixedly coupled to a second end 364 of the second portion 360 of the first band member 300. In another example, in some embodiments, the first portion 310 of the first band member 300 is integrally or monolithically formed with the second portion 360 of the first band member 300. A shoulder 350 defines a transition between the first portion 310 of the first band member 300 and the second portion 360 of the first band member 300. Shoulder 350 also defines one end of a range of motion for the first band member 300 and the second band member 400 relative to each other.

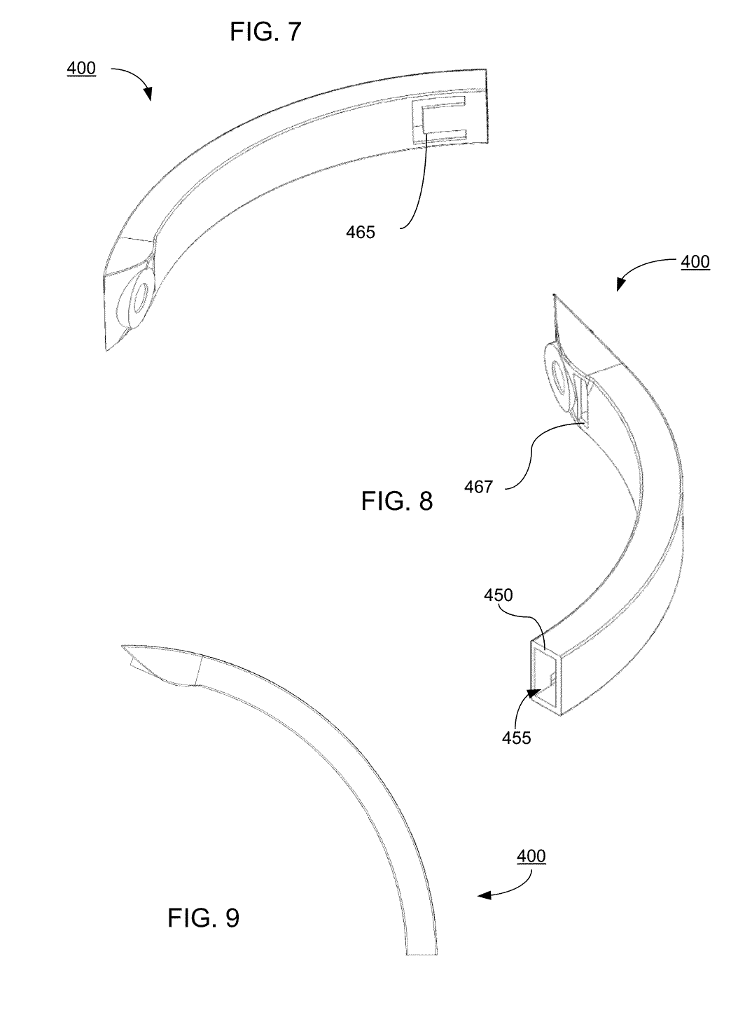

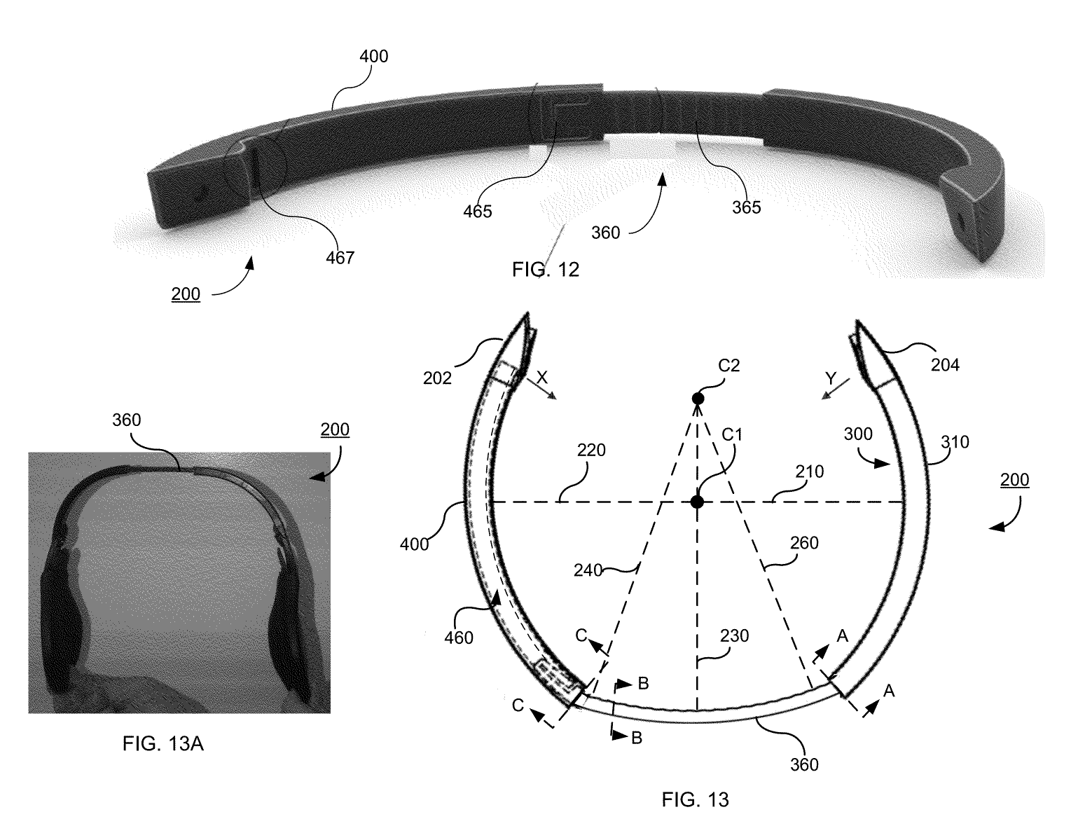

FIGS. 7-9 are views of the second band member 400 (i.e., in a first perspective view, a second perspective view and a top view, respectively). As shown in FIGS. 2 and 3, the second band member 400 can be slideably coupled to the first band member 300. An end surface 450 (see, e.g., FIG. 8) of the second band member 400 defines an opening 455 to a channel 460 (for example, as shown in dashed lines in FIGS. 11 and 13). The first band member 300 can be coupled to the second band member 400 by inserting the second portion 360 of the first band member 300 into the opening 455. Similarly stated, when the first band member 300 is coupled to the second band member 400, the second portion 360 of the first band member 300 is at least partially disposed within and slidably disposed within the channel 460. The first band member 300 can be coupled to the second band member 400 such that the second portion 360 of the first band member 300 is fully inserted into, or otherwise disposed within, the channel 460 of the second band member 400.

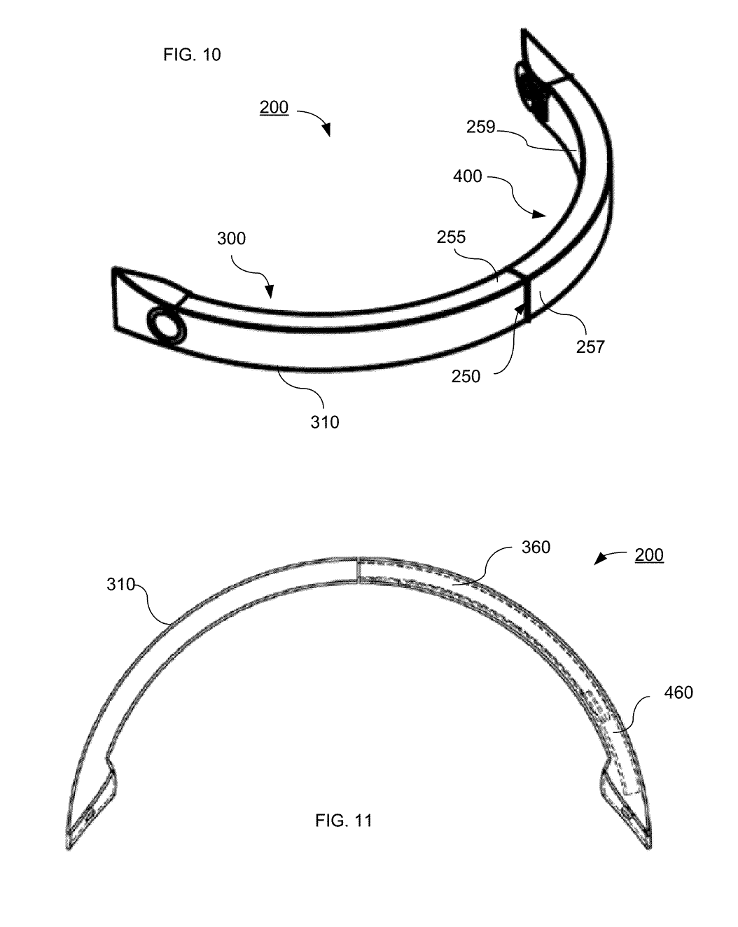

FIGS. 10 and 11 are views of the band 200 in the collapsed configuration. The first portion of the first band member 300 abuts the second band member 400 at the abutment interface 250. As shown, the band 200 is substantially rectangular, having a top face 255, an outer face 257, an inner face 259, and a bottom face (not shown in FIGS. 10 and 11). When the band 200 is in the collapsed configuration, each of the top face 255, outer face 257, inner face 259 and bottom face (collectively referred to as “faces”) form substantially continuous surfaces at the abutment interface 250. Similarly stated, the faces of the first band member 300 of the band 200 and the faces of the second band member 400 of the band 200 substantially align when the band 200 is in the collapsed configuration such that the surfaces of the faces are smooth at the abutment interface 250 and there are no substantial gaps, cracks, steps, or discontinuities of the surfaces of the faces at the abutment interface 250. For example, any gaps, cracks, steps, and/or discontinuities at the abutment interface 250 can be attributed to manufacturing tolerances and/or any gaps, cracks, steps, and/or discontinuities can have a size of less than 1 mm, less than 0.5 mm, less than 0.1 mm, etc.

As shown in FIG. 11, the second portion 360 of the first band member 300 can be disposed completely within the channel 460 of the second band member 400 when the band 200 is in the collapsed configuration. Similarly stated, the channel 460 can have a length at least as long as the second portion 360 of the first band member 300. Furthermore, when the band 200 is in the collapsed configuration, the abutment interface 250 can be substantially centered with respect to the minimum length of the band 200. Similarly stated, the second band member 400 can have a length substantially equal to the length of the first portion 310 of the first band member 300.

Furthermore, the opening 455 and/or channel 460 can be completely defined by the second band member 400. Similarly stated, the channel 460 can be completely internal to and/or surrounded on all sides by the second band member 400. In this way, the second portion 360 of the first band member 300 can be completely enveloped by the second band member 400 when the band 200 is in the collapsed configuration. Thus, in some embodiments, when the band 200 is in the collapsed configuration, no portion of the second portion 360 of the first band member 300 is exterior to, extends from, or is disposed outside of the second band member 400.

FIG. 12 is a view of the band 200 in the expanded configuration. In some embodiments, the second portion 360 of the first band member 300 includes ridges 365 or projections (see also, e.g., FIG. 5). As shown in FIG. 12, the ridges 365 are disposed along a length of the second portion 360 of the first band member 300. In some other embodiments, the ridges are disposed along a portion, but not all, of the length of the second portion of the first band member. The ridges 365 can be operable to increase friction between the first band 300 and the second band 400. Similarly stated, when the first band member 300 is moved relative to the second band member 400, the ridges 365 can contact a snap 465 (and/or other surfaces) of the second band member 400 producing a friction force tending to resist a change in length of the band 200. The snap 465 can be biased towards the channel 460 such that the snap applies a force inwardly towards the ridges 365. The friction force produced by the ridges 365 and/or the snap 465 can be sufficient to prevent the length of the band 200 from changing accidentally or when the frame is worn on the head, but can be low enough to allow a user to deliberately change the length of band 200. For example, the ridges 356 and/or the snap 465 can be operable to require a force of approximately 1 lb, approximately 3 lbs, approximately 5 lbs, and/or any other suitable force to overcome the friction force to slide the first band member 300 relative to the second band member 400, or vice versa.

In some embodiments, the snap 465 can include one or more ridges or projections (not shown) operable to interface with the ridges 365. For example, ridges on the snap 465 can be operable to define a ratcheting effect when the first band member 300 is moved relative to the second band member 400. The band 200 can include any suitable adjustment mechanism, such as any of the adjustment mechanisms shown and described herein, in U.S. Pat. No. 6,735,784, issued May 18, 2004; U.S. Pat. No. 6,978,483, issued Apr. 9, 2004; U.S. Pat. No. 7,996,923, issued Aug. 16, 2011; and/or U.S. patent application Ser. No. 13/209,603, filed Aug. 15, 2011, each entitled “Apparatus and Method for Making an Ear Warmer and an Ear Warmer Frame,” the disclosure of each of which is incorporated herein by reference in their entirety, or any suitable combination thereof.

The second band member 400 defines an opening 467 or vent. For example, as shown in FIG. 12, the inner face 259 of the second band member 400 defines the opening 467. In some embodiments, the opening 467 is disposed at a second end portion 414 of the second band member 400 on the band's 200 inner face 259. The opening 467 can be operable to allow air displaced by the second portion 360 of the first band member 300 to enter and/or exit the channel 460 when the length of the band 200 is adjusted. In this manner, the opening 467 is operable to substantially prevent a vacuum from being produced within channel 460 during adjustment of the length of the band 200 (e.g., when moving the band 200 from its collapsed configuration towards its expanded configuration), which could interfere with further adjustment of the band 200.

The first band member 300 can include a retaining clip 367 (see, e.g., FIG. 5). In some embodiments, retaining clip 367 is configured to contact the snap 465, thereby resisting or preventing the band 200 from further extension. In this way, the snap 465 and the clip 367 can resist or prevent the first band member 300 from inadvertently being removed from the channel 460, such as when the band 200 is adjusted to its maximum length in its expanded configuration. Similarly stated, once the band 200 is assembled, the retaining clip 367 and the snap 465 can collectively prevent the band from being disassembled and define one end of a range of motion for the first band member 300 and the second band member 400 relative to each other.

In some embodiments, the clip 367 is biased towards a first position, in which the clip 367 contacts the snap 465 when the band 200 is in its expanded configuration. The clip 367 is configured to be moved towards an outer face of the second portion 360 of the first band member 300 (e.g., by a squeezing or pressing force), such that the clip 367 no longer contacts the snap 465 and the first member 300 and second member 400 of the band 200 can be uncoupled, for example, if the user intends to disassemble the band members.

In the collapsed configuration, a portion of the first band member 300 and a portion of the second band member 400 are substantially circular and/or concentric (see, e.g., FIG. 11). Similarly stated, at least a portion of the band 200 including the first member 300 and the second member 400 can have a substantially uniform radius of curvature when the band 200 is in the collapsed configuration. As the band 200 is moved towards its expanded configuration, at least a portion of the first band member 300 and/or a portion of the second band member 400 is configured to flex or otherwise slightly deform such that at least a portion of the band is no longer substantially circular and/or concentric with other portions of the band 200. For example, as shown in FIG. 13, which is a bottom view of the band 200, a radius of curvature 210 associated with a first point of the first portion 310 of the first band member 300 and a radius of curvature 220 associated with a first point of the second band member 400, each measured from center point C1, can be substantially equal when the band 200 is in the expanded configuration. While a radius of curvature 230 associated with a first point of the second portion 360 of the first band member 300, and a radius of curvature 240 associated with a second point of the second portion 360 of the first band member 300, and a radius of curvature 260 associated with a third point of the second portion 360 of the first band member 300, measured from center point C2, are greater than the radii of curvature 210, 220 of the first portion 310 of the first band member 300 and the second band member 400, respectively, when the band 200 is in the expanded configuration. Said another way, and as described in more detail herein, the second portion 360 of the first band member 300 can deform, or flatten, as the band 200 is moved to its expanded configuration, thereby changing the radius of curvature of one or more points along a length of the second portion 360 of the first band member 300 relative to one or more points along a length of the first portion 310 of the first band member 300 and/or the second band member 400. Furthermore, the length of the second member 400 can be substantially equal to the length of the first portion 310 of the first member 300. In this way, as the arc length of the band 200 changes, the band can be substantially symmetrical.

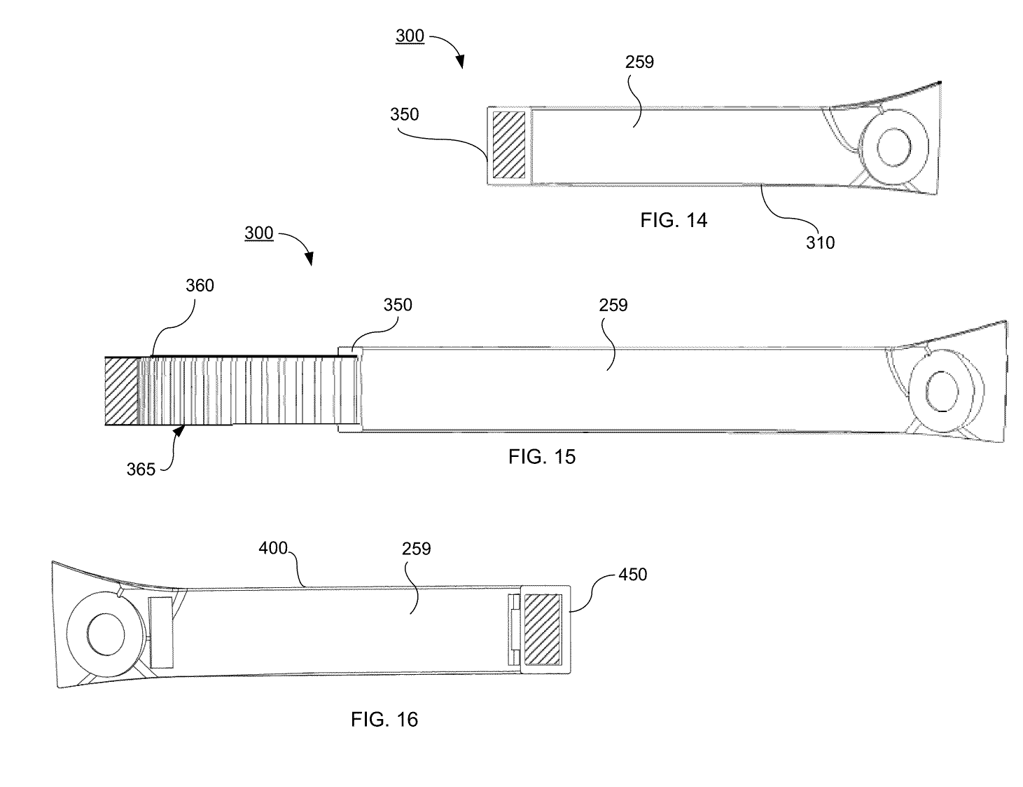

FIG. 14 is a cross-sectional view of the first portion 360 of the first member 300 taken along line A-A in FIG. 13 at the shoulder 350. FIG. 16 is side view of the second member 400 taken along line C-C in FIG. 13 showing end surface 450. A cross-sectional profile of the shoulder 350 of the first member 300 is substantially the same size and shape of the end surface 450 of the second band member 400. As shown, for example, in FIGS. 10 and 11, when the band 200 is in the collapsed configuration, the end surface 450 can abut the shoulder 350 at the abutment interface 250. In embodiments where the shoulder 350 is substantially the same size and shape as the end surface 450 (e.g., having a perimeter with substantially similar dimensions) substantially continuous surfaces can be defined when the band is in the collapsed configuration.

FIG. 15 is a cross-sectional view of the second portion 360 of the first band member 300 taken along line B-B in FIG. 13. The cross-sectional surface 361 of the second portion 360 of the first band member 300 has a size and shape smaller than the shoulder 350. The size and shape of the cross-sectional surface 361 corresponds to and is slightly smaller (e.g., less than 0.5 mm smaller than) the size and shape of the opening 455. Because the second portion 360 of the first band member 300 has a smaller cross-sectional area than each of the first portion 310 of the first band member 300 and the second band member 400, the second portion 360 of the first band member 300 can be more flexible than either of the first portion 310 of the first band member 300 or the second band member 400.

In some embodiments, when the band 200 is in the expanded configuration, the band 200 can be operable to be deformed, for example, from the substantially circular configuration shown in FIG. 11 to the curved non-circular configuration shown in FIG. 13 (and/or a configuration in which the second portion 360 of the first band member 300 is substantially flattened, as shown in FIG. 13A). In the collapsed configuration, the band 200 can be in an unbiased configuration. In the expanded configuration, the band 200 can be in a biased configuration. In the biased configuration, opposing ends 202, 204 of the band are inwardly biased such that the ends 202, 204 tend to move toward and/or apply a force in the direction of the arrows X and Y shown in FIG. 13. More specifically, in some embodiments, the ends 202, 204 of the band 200 are biased towards a center point C around which the band 200 is disposed. For example, the band 200 can be flexible to stretch the band 200 to fit around a head of a user, and biased such that the ends 202, 204 of the band 200 apply a light force against the head of the user such that the band 200 tends to be secured about the head of the user.

When the band 200 is biased, one or more radius of curvature (e.g., radii of curvature 230, 240, 260) of the second portion 360 of first band member 300 can increase, as described above with respect to FIG. 13. In some embodiments, the radius of curvature 230, 240, 260 of the second portion 360 first band member 300 can increase more than one or both of the radius of curvature 210 of the first portion 310 of the first band member 300 and the radius of curvature 220 of the second band member 400. Similarly stated, an intermediate portion of the frame 200 including the second portion 360 of the first band member 300 and at least a portion of the first portion 310 of the first member and at least a portion of the second band member 400 can transition from being substantially circular and/or having a substantially uniform radius of curvature to having a non-uniform radius of curvature. The radius of curvature of the first portion 310 of the first band member 300 can be greater when the band 200 is in the expanded configuration than when in the collapsed configuration. Similarly, the radius of curvature of the second band member 400 can be greater when the band 200 is in the expanded configuration than when in the collapsed configuration.

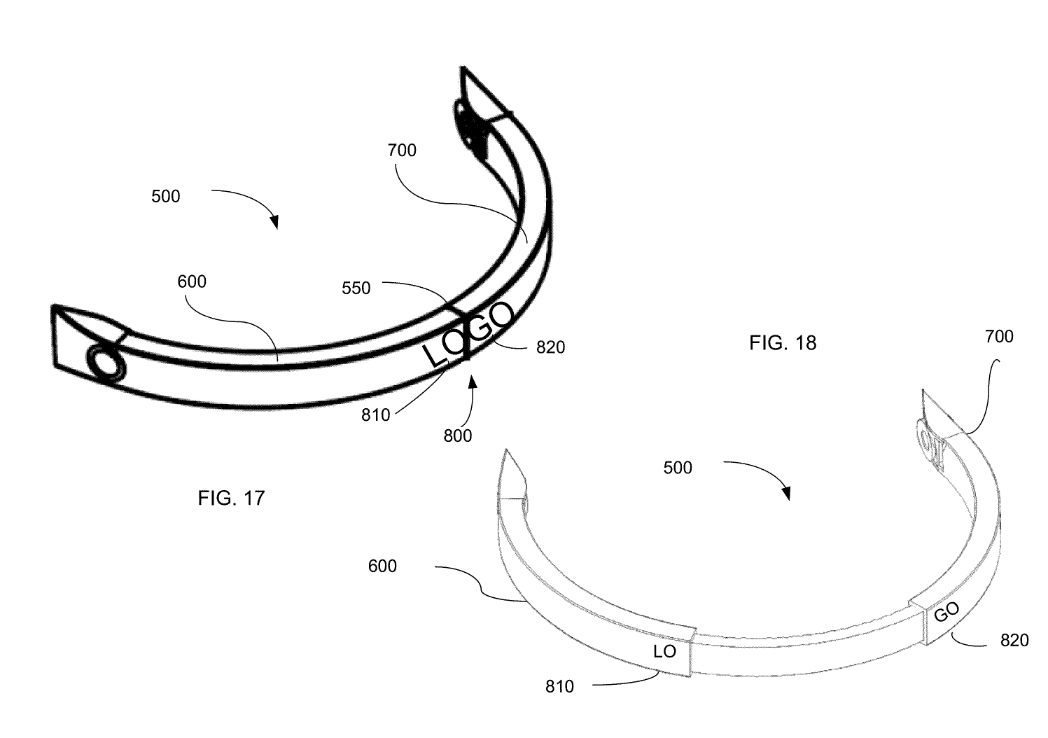

FIGS. 17 and 18 are perspective views of a band 500 in collapsed configuration and an expanded configuration, respectively. The band 500 can be structurally and/or functionally similar to the band 200 shown and described above. For example, the band 500 can include a first band member 600 structurally and/or functionally similar to the first band member 300 and a second band member 700 structurally and/or functionally similar to the second band member 400. The first band member 600 includes a first portion 810 of a logo 800 and the second band member 700 includes a second portion 820 of a logo 800. When the band is in the collapsed configuration, as shown in FIG. 17, the unified logo 800 appears on a substantially continuous surface about and including an abutment interface 550. The band 500 is substantially symmetrical, which results in the logo 800 appearing in the center of the band 500 in both the expanded configuration and the collapsed configuration as well as in any intermediate configurations.

While various embodiments of the invention have been described above, it should be understood that they have been presented by way of example only, and not limitation. Where any methods described herein indicate certain events occurring in certain order, the ordering of certain events may be modified. Additionally, certain of the events may be performed concurrently in a parallel process when possible, as well as performed sequentially as described above. Thus, the breadth and scope of the invention should not be limited by any of the above-described embodiments. While the invention has been particularly shown and described with reference to specific embodiments thereof, it will be understood that various changes in form and details may be made.

For example, although the band 200 is shown and described as having a substantially rectangular cross-sectional profile and having four surfaces (e.g., surfaces 255, 257, 259), in other embodiments, an ear warmer frame can include a band having any shape and/or any number of surfaces. For example, the band can have a cross-sectional profile that is oval or round (e.g., with a single continuous outer surface), octagonal, hexagonal, or any other suitable shape or combination of shapes. Furthermore, although four surfaces (e.g., surfaces 255, 257, 259) of a substantially rectangular band (e.g., band 200) are shown and described as being substantially continuous across the abutment interface, in other embodiments, any number of surfaces can be substantially continuous across the abutment interface. For example, one, two or three surfaces of a band having a substantially rectangular cross-sectional profile can be substantially continuous across the abutment interface 250 (e.g., such that one, two or three surfaces of the band include a discontinuity at the abutment interface). In an embodiment in which the band has a non-rectangular cross-sectional profile (e.g., oval, round, octagonal, hexagonal, etc., as described above), any number of surfaces can be substantially continuous across the abutment interface 250.

In another example, although the channel 460 is shown and described as being completely defined by the second band member 400, in other embodiments, a channel can be only partially defined by the second band member. For example, at least a portion of the second band member can define an elongate opening (and/or other opening) in fluid communication with the channel 460. Such an elongate opening can be in addition to, or in lieu of the opening 467 defined by the second band member 400. For example, such an elongate opening in communication with the channel 460 can be configured to permit the passage of air therethrough (i.e., into and/or out of the channel) as the band 200 is adjusted from its collapsed configuration to its expanded configuration or vice versa. Similarly, although the second portion 360 of the first member 300 is shown and described as being configured to be disposed entirely within the second band member 400 when the band 200 is in the collapsed configuration, in other embodiments, the second portion 360 of the first band member 310 may not be disposed entirely within the second band member 400 when the band 200 is in the collapsed configuration. For example, the second band member 400 can define an opening (e.g., an elongate or other shaped opening) on a side surface of the second band member 400 through which a portion of the second portion 360 of the first band member 300 can be exposed and/or through which a portion of the second portion 360 of the first band member 300 can protrude.

In another example, although the band 200 has been shown and described herein as being substantially circular or concentric in its collapsed configuration, in other embodiments, the band can have any suitable shape in its collapsed configuration. For example, the band can be non-circular or non-concentric (e.g., substantially U-shaped) in its collapsed configuration. Furthermore, although the band 200 has been shown and described herein as being unbiased in the collapsed configuration, in some embodiments, the band is biased in its collapsed configuration. For example, the band can be biased towards a coiled or other suitable configuration. In still another example, although the band 200 has been shown and described herein as having points with varying radii of curvature when the band is in the expanded configuration, in some embodiments, all or substantially points along a length of the band in the expanded configuration can have equal radii of curvature.

Although the channel is shown and described as being completely defined by the second band member, in other embodiments, the channel can be only partially defined by the second band member. Similarly, although the second portion of the first member is shown and described as to be disposed entirely within the second band member when the band is in the collapsed configuration, in other embodiments, the second portion of the first band member may not be disposed entirely within the second band member when the band is in the collapsed configuration. For example, the second band member can define an opening on a side surface through which a portion of the second portion of the first band member can be exposed and/or through with a portion of the second portion of the first band member can protrude.

Although the channel 460 and second portion 360 of the first member 300 have each been shown and described as having a substantially constant cross-sectional profile along their respective lengths, in other embodiments, a cross-sectional profile of the channel and/or the second portion of the first member can vary along its respective length. For example, a cross-sectional profile of the channel and/or the second portion of the first member can be tapered. In some other embodiments, the cross-sectional profile of each of the channel and the second portion of the first member can be tapered such that the tapered profile of the channel is complementary to the tapered profile of the second portion of the first member.

Although the second portion 360 of the first member 300 has been shown and described herein as including a clip 367 configured to prevent the accidental uncoupling (or disassembly) of the first member 300 from the second member 400, in other embodiments, an ear warmer band can include a different mechanism to prevent accidental uncoupling of a first member and a second member. For example, in some embodiments, the second portion of the first member can include a button or other protrusion configured to mate with a recess or opening defined by a face of the second member to prevent further movement of the first member away from the second member due to low levels of force, as described herein. In such embodiments, intentional uncoupling of the first member and the second member can still be possible, such as with the intentional application of a greater level of force to move the first member away from the second member.

Although various embodiments have been described as having particular features and/or combinations of components, other embodiments are possible having a combination of any features and/or components from any embodiment as discussed above. For example, band 200 can include the ear warmers 190 of band 100, shown and described with respect to FIG. 1, coupled to the first band member 300 and second band member 400, respectively. In another example, the band 200 can include the logo 800 of band 500, as shown and described with respect to FIGS. 17-18. In still another example, a first portion of a first band member of the band 500 can include the ridges 365 of the first portion 310 of the first band member 300 of the band 200.

What is claimed is:

1. An apparatus, comprising:
a frame configured to be disposed about a portion of a head of a user, the frame including:
a first member having a first portion and a second portion, the first portion of the first member having a cross-sectional profile, the second portion of the first member having a cross-sectional profile smaller than the cross-sectional profile of the first portion of the first member; and
a second member, an end portion of the second member having a cross-sectional profile substantially corresponding to the cross-sectional profile of the first portion of the first member, the second member defining an opening configured to receive the second portion of the first member such that when the frame is in a collapsed configuration an abutment interface is defined, surfaces of the abutment interface being substantially continuous between the first member and the second member.

2. The apparatus of claim 1, wherein the opening is completely defined by the second band portion.

3. The apparatus of claim 1, wherein the opening defines a channel, the second portion of the first member configured to be fully disposed within the channel when the frame is in the collapsed configuration.

4. The apparatus of claim 1, wherein the opening defines a channel, a surface area of the channel completely defined by the second band portion.

5. The apparatus of claim 1, wherein:
the first member includes a shoulder disposed between the first portion of the first member and the second portion of the first member; and
an end surface of the second band portion defines the opening and the cross-sectional profile of the end portion of the second member, the shoulder and the end surface configured to form the abutment interface when the frame is in the collapsed configuration.

6. The apparatus of claim 1, wherein:
the first member includes a first part of a logo; and
the second member includes a second part of the logo such that a continuous surface of the abutment interface includes the logo when the frame is in the collapsed configuration.

7. The apparatus of claim 1, wherein:
the first member includes a first part of a logo; and
the second member includes a second part of the logo such that the first part of the logo and the second part of the logo are separated when the frame is in an expanded configuration, and the first part of the logo and the second part of the logo are abutting on a surface of the abutment interface from the surfaces that are substantially continuous when the frame is in the collapsed configuration.

8. The apparatus of claim 1, wherein the second portion of the first member and a portion of the second member have a substantially common and substantially uniform radius of curvature when the frame is in the collapsed configuration.

9. The apparatus of claim 1, wherein (1) the second portion of the first member and (2) a portion of the second member have a length equal to a length of the second portion of the first member and collectively define an intermediate portion of the frame, the intermediate portion having a first arc length when the frame is in the collapsed configuration, the intermediate portion having a second arc length longer than the first arc length when the intermediate portion is in the expanded configuration.

10. An apparatus, comprising:
a frame configured to be disposed about a portion of a head of a user, the frame including:
a first member having a first portion, a second portion, and a shoulder disposed between the first portion and the second portion; and
a second member having an end surface defining an opening to a channel, the channel completely defined by the second band portion, the second portion of the first member configured to be fully disposed within the channel when the frame is in a collapsed configuration such that the shoulder abuts the second member, a perimeter of the shoulder substantially corresponding to a perimeter of the end surface such that a substantially continuous transition is formed between the first member and the second member when the frame is in the collapsed configuration.

11. The apparatus of claim 10, wherein the second portion of the first member and a portion of the second member that contains the channel collectively define an intermediate portion of the frame having a substantially uniform radius of curvature when the frame is in an unbiased configuration.

12. The apparatus of claim 11, wherein the second portion of the first member is configured to deform when the frame is moved from the unbiased configuration to a biased configuration such that the intermediate portion does not have a uniform radius of curvature when the frame is in the biased configuration.

13. The apparatus of claim 10, wherein the shoulder has a cross-sectional area greater than a cross-sectional area of the second portion of the first member.

14. The apparatus of claim 10, wherein a length of the second member is substantially equal to half a length of the frame when the frame is in the collapsed configuration.

15. An apparatus, comprising:
a frame configured to be disposed about a portion of a head of a user, the frame including:
a first member having a first portion, a shoulder, and a second portion, the shoulder disposed between the first portion and the second portion, the second portion having a substantially uniform radius of curvature when the frame is in a collapsed configuration; and
a second member having an end surface defining an opening to a channel, a portion of the second member having (1) a length substantially equal to a length of the second portion of the first member and (2) a substantially uniform radius of curvature substantially equal to the uniform radius of curvature of the second portion of the first member when the frame is in the collapsed configuration,
the shoulder abutting the end surface when the frame is in the collapsed configuration, a perimeter of the shoulder substantially corresponding to a perimeter of the end surface such that the frame substantially continuously transitions from the first member to the second member when the frame is in the collapsed configuration.

16. The apparatus of claim 15, wherein a substantially circular intermediate portion of the frame defined by the second portion of the first member and the portion of the second member has a first arc length when the frame is in a collapsed configuration and a second arc length longer than the first arc length when the frame is in the expanded configuration.

17. The apparatus of claim 15, wherein an intermediate portion of the frame defined by the second portion of the first member and the portion of the second member has a substantially circular shape when the frame is in an unbiased configuration.

18. The apparatus of claim 15, wherein the second portion of the first member is configured to deform when the frame is moved from the unbiased configuration to a biased configuration.

19. The apparatus of claim 15, wherein the second portion of the first member is configured to deform such that the intermediate portion is non-circular when the frame is moved from the unbiased configuration to a biased configuration.

20. The apparatus of claim 15, wherein the second portion of the first member has a cross-sectional area less than a cross-sectional area of the first portion of the first member and less than a cross-sectional area of the second member.

21. The apparatus of claim 15, wherein the second portion of the first member is configured to deform such that the second portion of the first member does not have a uniform radius of curvature when the frame is in an expanded configuration.

22. The apparatus of claim 15, wherein the second portion of the first member has a cross-sectional area less than a cross-sectional area of the first portion of the first member and less than a cross-sectional area of the second member such that the second portion of the first member is configured to deform more than the first portion of the first member and more than the second member when the frame is moved from an unbiased configuration to a biased configuration.
